(19) United States
(12) Patent Application Publication
(10) Pub. No.: US 2018/0369602 A1
(43) Pub. Date: Dec. 27, 2018

(54) LOW-LEVEL LASER LIGHT THERAPY DEVICES, AND METHODS OF USE

(71) Applicant: TICHAUER TECHNICAL LABORATORIES, LLC, La Palma, CA (US)

(72) Inventor: LARRY MARTIN TICHAUER, LA PALMA, CA (US)

(21) Appl. No.: 15/961,515

(22) Filed: Apr. 24, 2018

Related U.S. Application Data
(60) Provisional application No. 62/523,359, filed on Jun. 22, 2017.

(51) Int. Cl. A61N 5/06; H05B 33/08; A01G 7/04

(52) U.S. Cl. CPC A61N 5/06; H05B 33/0845; H05B 33/0815; A61N 5/0616; A61N 5/0618; A61N 2005/067; A61N 2005/0629; A61N 2005/0652; A61N 2005/0661; A61N 2005/0662; A01G 7/045

(57) ABSTRACT

Devices and methods for low-level laser light therapy for animals and plants are provided. A device can include a pulse generator configured to generate a pulse train, wherein a pulse wave of the pulse train is configured to have a rectangular, non-sinusoidal waveform and a duty cycle within a range of 0.5% and 20%, an emitter array comprising a plurality of light emitting diodes (LEDs) and in electrical connection with the pulse generator, a computer-readable medium comprising stored instructions which, when executed, cause an LED to emit light, and a housing unit.

### CROSS-REFERENCE TO A RELATED APPLICATION

This application claims the priority benefit of U.S. Provisional Application Ser. No. 62/523,359, filed Jun. 22, 2017, which is incorporated herein by reference in its entirety.

### BACKGROUND OF THE INVENTION

Low-level laser light therapy (LLLLT) is an alternative form of medicine that has enjoyed widespread use in Europe and Asia prior to reaching an audience in the United States. Electromagnetic waves within a wavelength range of 632 nm to 1064 nm can transport energy through the epidermal and dermal skin layers and deliver said energy to animal and plant tissue to stimulate cell metabolism and reduce inflammation. Among the many beneficial therapeutic effects, LLLLT can provide a non-invasive therapeutic treatment for pain relief, hair growth, and tissue damage.

Alternative forms of medicine, such as LLLLT, are gaining acceptance in the medical community in the United States. As more information is made available and processed, patients are recognizing the inherent health and cost benefits of alternative medicine options, which reduce or eliminate the necessity of drug treatment regimens or surgical procedures. American consumers are increasingly using alternative forms of medicine and as insurance providers begin to cover alternative medicinal treatments, these figures are expected to grow. Rising consumer demand and shifting treatment priorities create the need for new and novel devices and methods to satisfy consumer demand in the alternative health care market.

Traditional LLLLT techniques rely on lasers and light emitting diodes (LEDs) that consume a significant amount of power. Among the causes for substantial power consumption are signal processing decisions, design choices, and electronic component requirements. A need exists for LLLLT devices and methods that can provide therapeutic effects while consuming less power.

### BRIEF SUMMARY OF THE INVENTION

The subject invention provides LLLLT devices and methods that provide patients with alternative medicinal treatment and reduce power consumption over similar devices. Advantageously, the devices and methods use high frequency signals with a small duty cycle to effectuate cellular regeneration, reduce inflammation in a human or animal body, and operate with low power consumption.

The present invention further provides devices and methods that stimulate cellular regeneration and photosynthesis in plants.

The device and methods described herein can be used by dermatologists and/or patients either seeking alternative medicine therapy or supplementing traditional medical treatment regiments with LLLLT. For example, a patient may choose to use only LLLLT to treat an inflamed region of his/her body or apply LLLLT in conjunction with anti-inflammatory pain relieving drugs, such as aspirin or ibuprofen.

In one embodiment, an LLLLT device can be positioned at a predetermined distance away from a target region of the body. The distance can vary based upon the individual's condition and desired medicinal effect. The device can be further configured to emit a signal for a predetermined period of time or to respond to the user's manual input. The photons emitted from the LLLLT device contain energy and can pass through the epidermal and dermal layers of the skin to induce a photochemical reaction in the damaged tissue.

The LLLLT device of the subject invention can be used for a variety of treatments that include but are not limited to:

1) scar healing;

2) inflammation reduction;

3) respiratory ailments;

4) joint pain;

5) Irritable Bowel Syndrome (IBS);

6) excessive swelling;

7) back pains;

8) hair loss;

9) cataracts; and

10) ocular injury or retinal damage.

In another embodiment, the device of the subject invention is used to promote plant growth.

The devices described herein can be used to deliver LLLLT, are easy to use, and require low power consumption. Additionally, the device can be light-weight, portable, and mass produced at a minimal per-unit cost.

### BRIEF DESCRIPTION OF THE DRAWINGS

A better understanding of the features and advantages of the systems and methods provided will be obtained by reference to the following detailed description that sets forth illustrative embodiments and the accompanying drawings of which:

FIG. 1 shows a system block diagram of the device, according to an embodiment of the subject invention.

FIG. 2 shows a five LED emitter array, according to an embodiment of the subject invention.

FIG. 3 shows the front face of a portable LLLLT device, according to an embodiment of the subject invention.

FIG. 4 shows a portable LLLLT device with a LED array in a linear pattern, according to an embodiment of the subject invention.

FIGS. 5A-5G show an image of two stitched skin lesions. The upper lesion has had LLLLT delayed for a period of six days and the lower lesion has had LLLLT applied immediately after surgery.

### DETAILED DESCRIPTION OF THE INVENTION

The present invention provides devices and methods for therapeutic treatment of, for example, tissue damage, respiratory ailments, digestive issues, swelling from injury or inflammation, hair rejuvenation, muscular pains, eye injuries, and nerve related pain. Specifically, the subject invention provides devices and methods for efficient LLLLT that require low power consumption and reduce the healing time period of a patient.

More specifically, the present invention provides devices and methods to configure an LLLLT device with a small duty cycle, a high frequency, and a low pulse rate frequency (PRF) signal to generate electromagnetic waves that provide therapeutic relief.

In other embodiments, the device and methods configure an LLLLT device to emit waves at a wavelength that stimulates plant life growth and/or blooming.

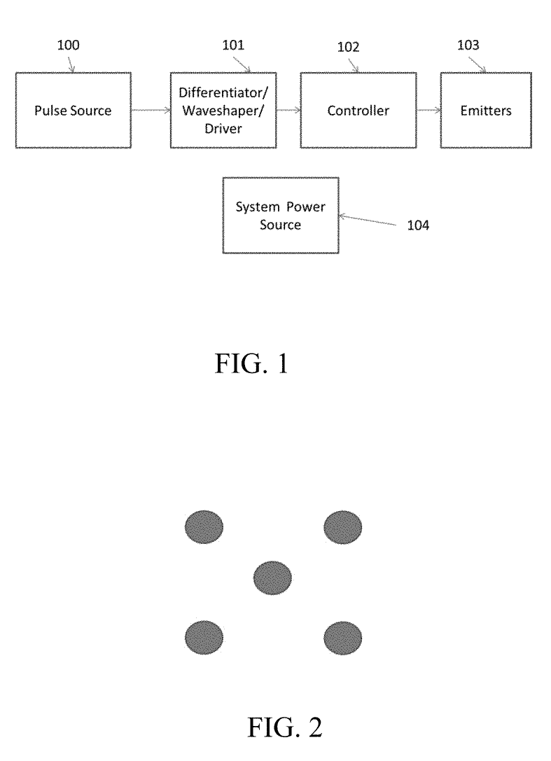

Referring to FIG. 1, there is shown a system block diagram of the synergistic light source which includes a pulse source 100, a differentiator/waveshaper/driver 101, a controller 102, an emitter 103, and a system power source 104. The pulse source 100 generates digital pulse signals, typically in the form of square or rectangular waves, and transmits the pulse signals to the differentiator/waveshaper/driver 101. The pulse source 100 may additionally have the capability to adjust the frequency, the pulse width, the voltage level, the rise times, and the fall times of the pulse signal. The differentiator/waveshaper/driver 101 can receive the signal from the pulse source, modulates the digital pulse, and transmit the signal to the controller 102. This step in the process can be used to ensure that the controller 102 only receives uniform digital pulses. In other embodiments, the controller 102 can receive non-uniform signals. The controller 102 receives the output signal from the differentiator/waveshaper/driver 101 and controls, manages, and directs the output signal to one or more peripheral device(s) which can be hardwired or wirelessly controlled. The controller 102 can be configured to transmit a predetermined signal for a predetermined time period or be manually adjusted during operation of the device. It should be appreciated by one of ordinary skill in the art that the controller 102 may be a component of a central processing unit (CPU). In an embodiment of this invention, the peripheral device (emitter 103) comprises an array of light emitting diodes (LEDs) with wide beam widths. The number and pattern of the emitters 103 can vary based upon the desired treatment and application.

The device can be powered by a system power source 104, which can include but is not limited to: a non-rechargeable battery, a rechargeable battery, an external power source connected to the device through a USB port, a wireless energy source, or solar power. In certain embodiments of the subject invention, an operator can enter instructions, including but not limiting to: duration of treatment, frequency, and duty cycle percentage, into a remote interface and wirelessly transmit the instructions to one or more emitter arrays that are spatially separate from the interface. It should be appreciated by those skilled in the art that the wireless signal can comprise a variety of instructions, including but not limited to frequency modulation, duty cycle modulation, and messaging.

In one embodiment, the differentiator comprises an RC differentiator. The RC differentiator is a high pass filter and can comprise a series capacitor connected in series to a shunt resistor terminated to a low impedance point such as ground or a bias voltage. The waveshaper receives the output signal from the RC differentiator, synthesizes the signal through a transfer function and emits an output signal. In certain embodiments, this output signal comprises a pulse wave or pulse train with a small duty cycle. Waveshaping can be a nonlinear process, in which an input signal at a first frequency f₁can be synthesized to produce an output signal. The output signal can comprise the sum of the first frequency f₁and integer multiples of the first frequency f₁. These additional frequencies contained within the output signal are known as harmonics. Subharmonics are signals at frequencies that are an integer submultiple of a frequency.

In one embodiment the differentiator/waveshaper/driver 101 can output a signal comprising the first frequency f₁and a harmonic or subharmonic of the first frequency f₁.

A duty cycle is the ratio of a pulse width to a pulse period and is often expressed as a percentage. The duty cycle of a signal can be derived from the following equation:

$$Duty Cycle = \frac{Pulse Width}{Pulse Period} \times 100$$

The duty cycle can be viewed as the percentage of time that the device is on while operating. In one embodiment, the pulse period is an integer submultiple of 15 μs. In preferred embodiments, the pulse period is approximately 15.13 μs. In other embodiments, the duty cycle of the pulse signal has a range of, for example, 0.1% to 20%, 0.5% to 10% or 1.5% to 3.5%. These ranges include any values within these ranges. In a preferred embodiment, the duty cycle has a target value of approximately 2.6%. A pulse repetition frequency (PRF) is the number of times a pulse is repeated within a predetermined unit of time. The PRF is measured in units of cycles/time period, in which the time period is usually one second and therefore the PRF units are then described in Hertz (Hz).

A low duty cycle of, for example, 0.5% to 20% correlates to a low percentage on time per cycle. The low duty cycle allows the device to operate with low power consumption, while still providing therapeutic benefit to the patients.

In one embodiment, the pulse source 100 and differentiator/waveshaper/driver 101 can be replaced with a pulse generator, microprocessor, or other device or devices capable of generating a pulse. In certain embodiments, either a pulse source 100 and differentiator/waveshaper/driver 101, a pulse generator, a microprocessor, or another device or devices are configured to emit a pulse train, in which each pulse of pulse train comprises a rectangular, non-sinusoidal waveform with a duty cycle range of 0.5% to 20%, and at a pulse repetition frequency range of 55 kHz to 70 kHz. In other embodiments, a device or devices are configured to emit a pulse at pulse repetition frequency of approximately 125 kHz to stimulate plant growth.

In one embodiment, the controller 102 can be configured to transmit a signal comprising a continuous pulse train to the emitters 103. In another embodiment, the controller 102 can be configured to allow either a caregiver or a patient to manually interrupt the signal for a period of time and restart the signal. In another embodiment, the controller 102 can be configured to transmit a predetermined number of pulses or transmit pulses for a predetermined period of time. In yet another embodiment, the controller 102 can be configured to transmit a predetermined number of pulses, which can be constant or a function of a predetermined period of time. In still yet another embodiment the controller 102 can be configured to allow a caregiver or patient to manually choose a constant or variable duty cycle. In yet another embodiment the controller 102 could drive a cluster of remote light emitters which could allow for coherent illumination of a given area. This area could be a room or an enclosed environment similar to a tanning fixture configuration.

Referring to FIG. 2, there is shown an emitter 103 array, according to one embodiment of the subject invention. In this embodiment, the emitter 103 array comprises four LEDs arranged in a square pattern with each emitter 103 disposed at a corner of the square pattern. A fifth LED is disposed at a center of the square pattern. In this embodiment, the emitter 103 array comprises a plurality of LEDs, which can vary in number and pattern depending upon the desired therapeutic effect. In this embodiment, the emitter 103 array will be arranged in an N×M array, wherein N and M can any value of one or greater. In this embodiment, at least one LED will emit a wave in the visible spectrum to convey to a user that the device is active.

In one embodiment of the subject invention, the device emits a signal within a pulse repetition frequency range of 55 kHz to 70 kHz. In a preferred embodiment, the device emits a signal at a pulse repetition frequency of 65 kHz. In another embodiment, the device emits a signal at a subharmonic pulse repetition frequency of 65 kHz. It should be appreciated by one of ordinary skill in the art that the device is capable of operating at pulse repetition frequencies up to 130 kHz. In one embodiment of the subject invention, the device operates at pulse repetition frequencies of 110 to 130 kHz, 120 to 130 kHz or at 125 kHz in order to stimulate plant growth. In one embodiment of the subject invention, at least one LED will be configured to emit waves with a wavelength of approximately 610 nm to 760 nm in order to produce red light.

It should be appreciated by a person of ordinary skill in the art that different rejuvenate and therapeutic benefits can be obtained through different frequencies and wavelengths within the electromagnetic spectrum. For example in an embodiment of the subject invention, at least one LED can be configured to emit waves with a wavelength within a range of approximately 495 nm to 577 nm, in order to produce green light to stimulate photosynthesis in plants. In another embodiment, at least one LED can be configured to emit waves with a wavelength within a range of approximately 450 nm to 494 nm in order to produce blue light. In another embodiment, at least one LED can be configured to emit waves with a wavelength within a range of approximately 280 nm to 400 nm in order to produce ultraviolent light. In yet another embodiment, the LED can be configured to emit waves with a wavelength within a range of approximately 840 nm to 940 nm to be outside the visible spectrum.

In another embodiment, plants can be simultaneously subjected to a combination of different wavelengths or different wavelengths during different periods of time. In yet another embodiment of the subject invention, at least one emitter of the emitter array can be configured to emit light at a first wavelength within a specified target range and another emitter can be configured to emit light at a second wavelength within a different target range. In certain embodiments of the subject invention, the electromagnetic waves can be configured to various frequencies and wavelengths in order to generate cytokine stimulation in a subject in need of therapy.

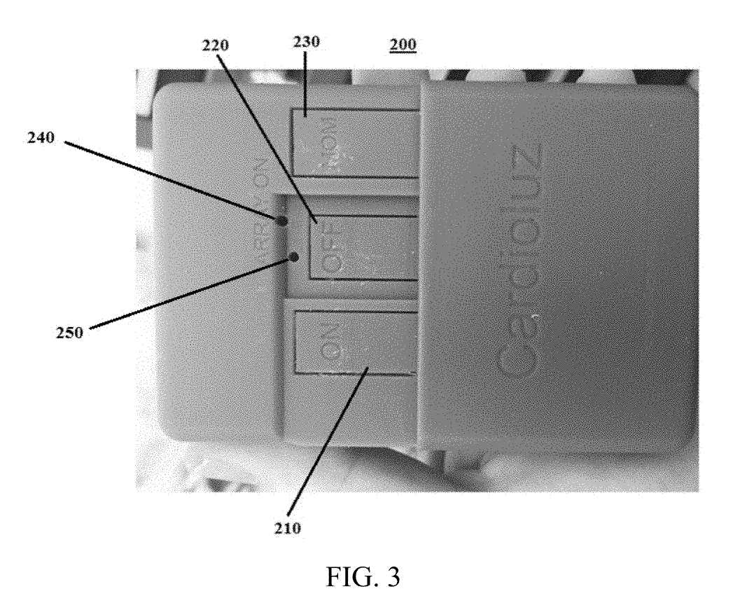

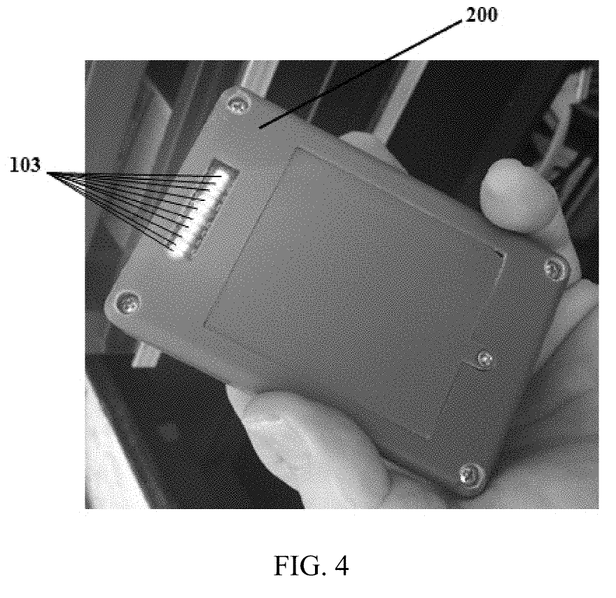

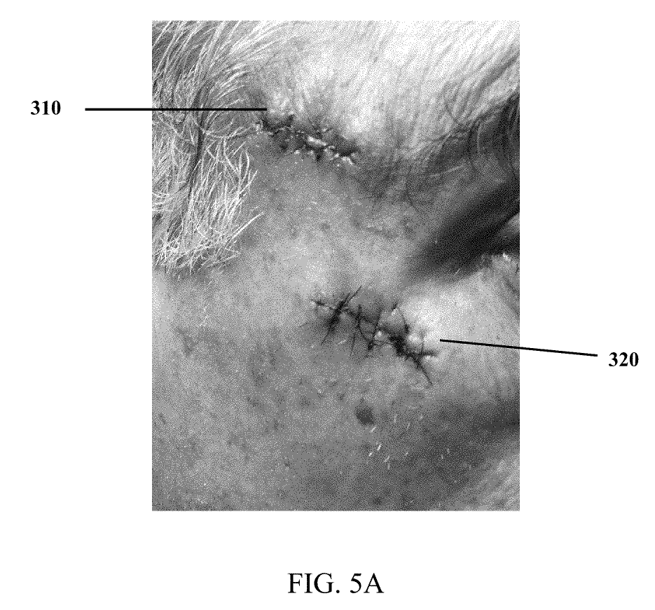

Referring to FIGS. 3 and 4, in this embodiment, the LLLLT device can comprise a portable, handheld, and battery powered device 200. Referring to FIG. 3 specifically, the device can include one face that comprises an “on” (ON) tab 210, an “off” (OFF) tab 220, a “momentary” (MOM) tab 230, an LED to indicate when the device is operating 240, and an LED to indicate the device is low on power 250. The “on” tab 210 can be configured to cause the device to operate until the “off” tab 220 is depressed. The “momentary” tab 230 can be configured to cause the LEDs to emit a signal when the tab is depressed and cease at the point that the “momentary” tab 230 is released.

In one embodiment, the LLLLT device can reduce hypertrophic scarring resulting from surgery. In order to maximize the results, the patient is exposed to daily treatment beginning within twenty-four hours of surgery. Swelling and inflammation reduction is apparent within days of beginning the LLLLT treatment. In preferred embodiments, LLLLT is commenced immediately after surgery, scarring is either minimal or non-existent after one month of treatment.

In one embodiment, keloid scar tissue can be reduced after a series of LLLLT treatments. Reduction of keloid scar tissue can occur even if treatment does not begin for a period of months after the keloid scar tissue initially surfaces.

In one embodiment, LLLLT can provide a non-invasive treatment for breathing and respiratory problems. A caregiver or patient can use the LLLLT device on sites that would be traditionally targeted during acupuncture therapy and provide therapeutic relief similar to that of lung acupuncture therapy by exposing the appropriate acupuncture points to the LLLLT device output. It should be understood that the LLLLT device and methods described herein are not limited to providing relief similar to lung acupuncture therapy and can be used to stimulate all acupuncture points in place of a needle to achieve the desired result based upon each acupuncture point position.

In one embodiment, LLLLT can be applied to sufferers of bone-to-bone knee issues and calcification in the knee. Users of the LLLLT have experienced increased levels of range of motion and reduction in pain and discomfort after a period of treatment.

In one embodiment, LLLLT can be applied to the abdominal region of a patient suffering from Irritable Bowel Syndrome (IBS). The treatment has been observed to decrease the symptoms of IBS and prolonged daily treatment eventually leads to longer periods of relief and longer periods of time in between treatments. In one embodiment, individuals with decreased or diminished bowel sounds due to stomach related ailments can benefit from LLLLT. These individuals have been observed to experience increased bowel sounds and have the ability to eat solid food earlier than patients who are only treated with traditional medicine.

In one embodiment, LLLLT can be used to decrease edema. The LLLLT device has been observed to stimulate mitochondrial activity and reduce edema.

In one embodiment, LLLLT is capable of, when used in conjunction with a treatment capable of neutralizing dihydrotestosterone, stimulating hair follicle rejuvenation and therefore allow for an increase in the hair follicle diameter, which will allow for a longer hair shaft.

In one embodiment, the LEDs can be configured, for example by modulating wavelengths, to simulate the seasons or respond to seasonal changes. For example, in certain embodiments, an LED of the emitter array can be configured to have increased light intensity during the winter months. In another embodiment, the LLLLT device can be configured to provide longer treatments in order to induce chemical stimulation during the winter months. In another embodiment an LED can be configured to have decreased or increased light intensity to provide therapeutic relief in response to a subject's increased exposure to sunlight during the summer months. In one embodiment, the intensity of light can be modulated as function of time and wavelength to account for a season dependent need of a subject. In yet another embodiment, LLLLT can be delivered in multiple stages in order to optimize the therapeutic relief to the subject in need of treatment. In yet another embodiment, the LLLLT device can be configured to emit light within the ultraviolet and infrared spectrum in order to optimize therapeutic benefits of therapy related to seasonal conditions or variations.

It should be appreciated by those skilled in the art that the intensity of light from an LED can be modulated through different methods including, but not limited to, Pulse Width Modulation (PWM) or current control. In one embodiment the therapeutic light from the LEDs can be directed to a subject in order to optically stimulate target regions of the body, including but not limited to the brain or nervous system.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that is capable of storing computer-readable information/data. Computer-readable media should not be construed or interpreted to include any propagating signals. A computer-readable medium of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

### EMBODIMENTS

Specific embodiments of the invention include the following:

Embodiment 1. A low-level laser light therapy (LLLLT) device, comprising:

a pulse generator configured to generate a pulse train,

wherein a pulse wave of the pulse train is configured to have a rectangular, non-sinusoidal waveform and a duty cycle within a range of 0.5% and 20%;

a central processing unit (CPU) connected the pulse generator;

an emitter array connected to the CPU,

wherein the emitter array comprises a plurality of emitters, in which each emitter is a light emitting diode (LED);

a power source delivering power to the device; and

a housing unit.

Embodiment 2. The device of embodiment 1, wherein the pulse wave of the pulse train has a duty cycle of 1.5% to 3.5%.

Embodiment 3. The device according to any of embodiments 1-2, wherein at least one emitter of the emitter array is configured to output a signal within a pulse repetition frequency range of 55 kHz to 70 kHz.

Embodiment 4. The device according to any of embodiments 1-3, wherein at least one emitter of the emitter array is configured to output a signal at a first pulse repetition frequency of 65 kHz.

Embodiment 5. The device according to any of embodiments 1-4, wherein at least one emitter of the emitter array is configured to output a signal at a second pulse repetition frequency, wherein the second frequency is a subharmonic of the first pulse repetition frequency of 65 kHz.

Embodiment 6. The device according to any of embodiments 1-5, wherein at least one emitter of the emitter array is configured to output a signal at a pulse repetition frequency of 125 kHz.

Embodiment 7. The device according to any of embodiments 1-6, wherein the pulse wave has a period of 15.13 μs.

Embodiment 8. The device according to any of embodiments 1-7, wherein the pulse wave has a period that is an integer multiple or submultiple of 15 μs.

Embodiment 9. The device according to any of embodiments 1-8, wherein at least one emitter of the emitter array is configured to emit light at a wavelength within a range of 450 nm to 494 nm to produce blue light.

Embodiment 10. The device according to any of embodiments 1-9, wherein at least one emitter of the emitter array is configured to emit light at a wavelength within a range of 280 nm to 400 nm to produce ultraviolent light.

Embodiment 11. The device according to any of embodiments 1-10, wherein at least one emitter of the emitter array is configured to emit light at a wavelength within a range of 495 nm to 570 nm to produce green light.

Embodiment 12. The device according to any of embodiments 1-11, wherein at least one emitter of the emitter array is configured to emit light at a wavelength within a range of 610 nm to 760 nm to produce red light.

Embodiment 13. The device according to any of embodiments 1-12, wherein at least one emitter of the emitter array is configured to emit light at a wavelength within a range of 840 nm to 940 nm to be outside the visible spectrum.

Embodiment 14. The device according to any of embodiments 1-13, wherein the emitter array comprises a first emitter and a second emitter, wherein the first emitter is configured to emit light at a first wavelength within a first specified range and the second emitter is configured to emit light at a second wavelength within a second specified range, wherein the first specified range is different than the second specified range.

Embodiment 15. The device according to any of embodiments 1-14, wherein the CPU is configured to direct a predetermined number of pulse waves to the emitter array.

Embodiment 16. The device according to any of embodiments 1-15, wherein the CPU is configured to direct the emitter array to emit a signal for a predetermined period of time.

Embodiment 17. The device according to any of embodiments 1-16, wherein the CPU is configured to transmit a signal to each LED of the emitter array, such that the signal transmitted to the LED of the emitter array is in phase with the signal transmitted to any other LED of the emitter array.

Embodiment 18. The device according to any of embodiments 1-17, wherein the emitter array comprises a plurality of LEDs, arranged in an N×M pattern, wherein N or M can be any value of 1 or greater.

Embodiment 19. The device according to any of embodiments 1-18, wherein at least one emitter of the emitter array is configured to emit visible light.

Embodiment 20. The device according to any of embodiments 1-19, wherein the emitter array comprises four LEDs arranged in a square pattern with each LED is disposed at a corner of the square pattern, and wherein a fifth LED is disposed at a center of the square pattern.

Embodiment 21. The device according to any of embodiments 1-20, wherein the housing unit has a first face, positioned on a first side of the device that comprises: an “on” switch configured to turn the device on; an “off” switch configured to turn the device off; a “momentary” switch configured to turn the device on when depressed and to turn the device off when released; a LED configured to emit visible light when the device is on and not emit visible light when the device is off; and a second face positioned on a second side, wherein the second face comprises a plurality of apertures configured to permit the emitter array to emit light.

Embodiment 22. The device of embodiment 21, wherein the second face is parallel to and on an opposite side of the first face.

Embodiment 23. The device of embodiment 21, wherein the second face is perpendicular to the first face.

Embodiment 24. The device according to any of embodiment 1-23, wherein the pulse generator comprises:

a pulse source;

an RC differentiator;

a waveshaper; and

a driver.

Embodiment 25. A low-level laser light therapy (LLLLT) device, comprising:

a first module comprising:

a control interface having a plurality of operable switches configured to produce an LED regulating signal,

wireless transmitter circuitry configured to encode the LED regulating signal and wirelessly transmit the encoded LED regulating signal; and

at least one second module comprising:

a pulse generator or repeater configured to generate a pulse train,

wherein a pulse wave of the pulse train is configured to have a duty cycle between 0.5% and 20%,

at least one emitter array,

wherein the at least one emitter array comprises a plurality of emitters, in which each emitter is an LED,

wherein at least one LED is configured to operate in response to the decoded LED regulating signal;

wireless receiver circuitry configured to receive the encoded LED regulating signal, decode the encoded LED regulating signal, and transmit the decoded LED regulating signal to the emitter array;

a housing unit to house the at least one emitter array; and

a power source delivering power to the device.

Embodiment 26. A method of applying low-level laser light therapy (LLLLT), said method comprising:

generating an output light from an LED of the device of claim 1;

positioning the device of claim 1 at a distance from a target region of a body of a subject in need; and

exposing the target region of the body of the subject in need to the output light.

Embodiment 27. The method of embodiment 26, wherein the device of claim 1 is configured to output light for a predetermined period of time.

Embodiment 28. The method according to any of embodiment 26-27, wherein the device of claim 1 is configured to output light for a time period that correlates to a predetermined number of pulse waves.

Embodiment 29. The method according to any of embodiment 26-28, wherein at least one emitter of the emitter array is configured to emit a wavelength within a range of 280 nm to 570 nm to stimulate cellular regeneration in plants.

Embodiment 30. The method according to any of embodiment 26-29, wherein the LLLLT is applied for the treatment of at least one of the following:

hypertrophic scarring;

keloid scar tissue;

breathing and respiratory problems;

bone to bone knee issues;

joint pain;

calcification of bone;

abdominal pain;

Irritable Bowel Syndrome;

edema;

hair loss;

Seasonal Affective Disorder;

sleep disorder;

sciatica;

cataracts; and

retinal damage or eye injury.

Embodiment 31. The method according to any of embodiments 26-30, wherein the device of claim 1 is configured to output light in order to simulate the seasons or in response to seasonal changes.

Embodiment 32. A method of promoting plant growth, wherein said method comprises:

generating an output light from an LED of the device of claim 1;

positioning the device of claim 1 at a distance from a target region of a subject plant in need; and

exposing the target region of the subject plant in need to the output light.

Embodiment 33. A method of promoting plant growth, wherein the method comprises:

outputting light at a pulse repetition frequency of 125 kHz from an LED of the device of embodiment 1;

positioning the device of at a distance from a target region of a subject plant in need; and

exposing the target region of the subject plant in need to the output light.

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The invention is described in greater detail by the following non-limiting examples.

### Example 1—Demonstration Of Therapeutic Effects Of LLLLT On Facial Scarring

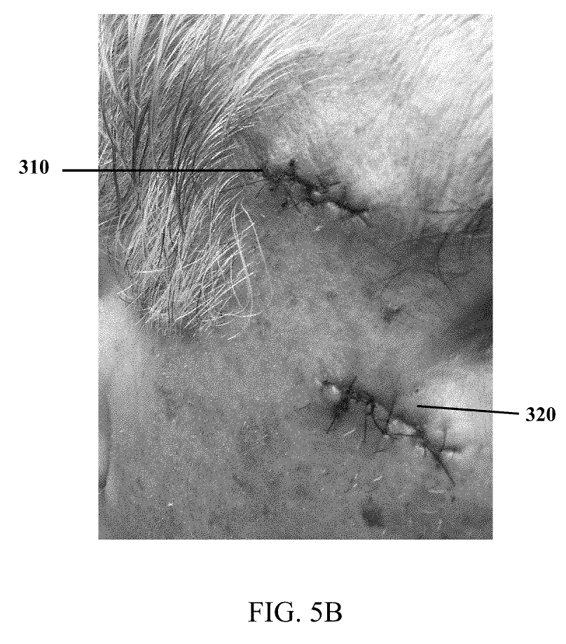

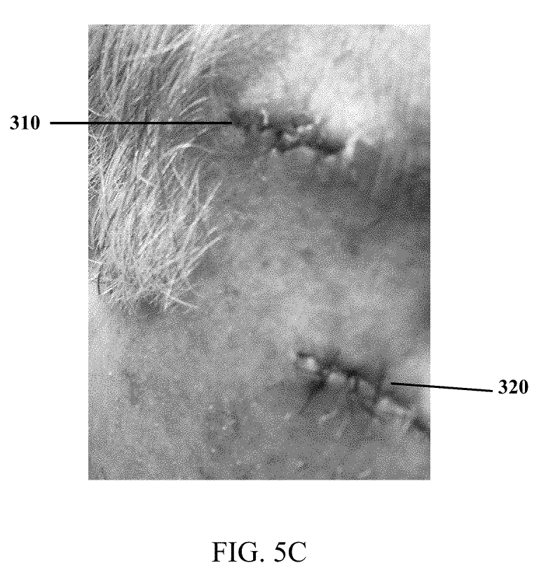

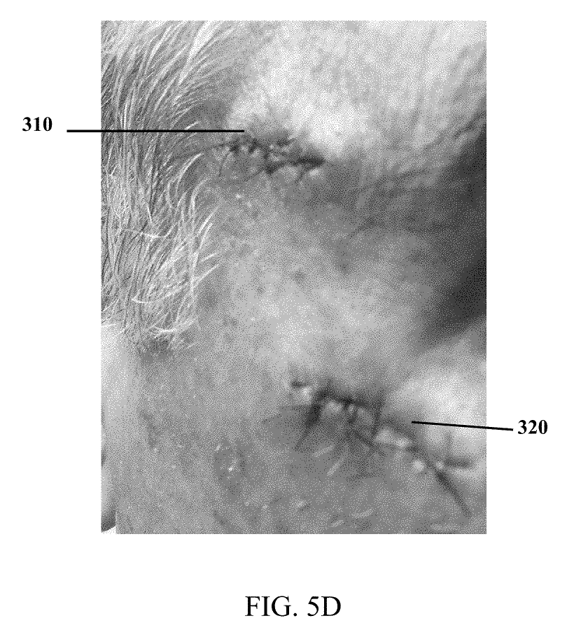

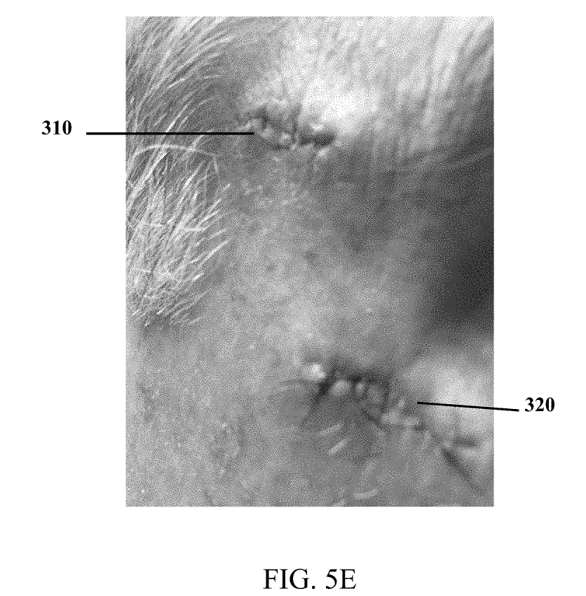

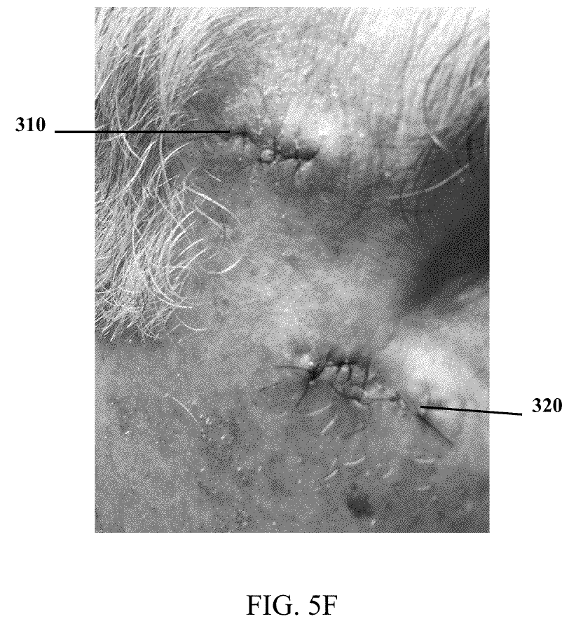

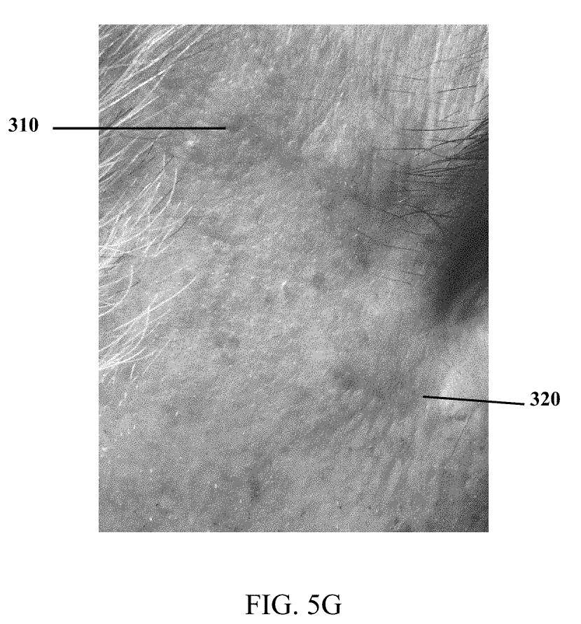

The healing rate of two closely located stitches has been observed. Referring to FIG. 5, the image of an upper stitch 310 and a lower stitch 320 can be seen above and below the right eye of a patient. LLLLT was directed to the lower stitch 320 for approximately three minutes per day for a period of one month beginning the day after surgery. Treatment on the upper stitch 310 was delayed for a period of six days and then applied equally with the lower stitch 320. LLLLT on the upper stitch 310 ended on the same day as the lower stitch 320.

FIGS. 5(a)-5(f) present images of the contrast in healing between the upper stitch 310 and the lower stitch 320 during the first six days after surgery. FIG. 5(a) presents an image of the upper stitch 310 and the lower stitch 320 one day after surgery. FIG. 5(b) presents an image of the upper stitch 310 and the lower stitch 320 two days after surgery. The lower stitch 320 appears to be healing quicker and swelling has been reduced. FIG. 5(c) presents an image of the upper stitch 310 and the lower stitch 320 three days after surgery. Without LLLLT, the upper stitch 310 still displays more pronounced swelling than the lower stitch 320. FIG. 5(d) presents an image of the contrast of the upper stitch 310 and the lower stitch 320 four days after the surgery. The lower stitch 320 displays reduced swelling and the wound is beginning to close. FIG. 5(e) shows that after five days after surgery, as seen, the lower stitch 320 continues to heal at an accelerated rate over the upper stitch 310. FIG. 5(f) shows that after six days after surgery, as seen, the lower stitch 320 still continues to heal at an accelerated rate over the upper stitch 310.

FIG. 5(g) shows an image of the upper stitch 310 and lower stitch 320 one month after the date of surgery. The upper stitch 310 presents hypertonic scarring, while the lower stitch 320 presents almost no scarring.

### Example 2—Demonstration Of Therapeutic Effects Of LLLLT On Eye Injury

A subject's right eye was injured due to exposure to a dose of ultraviolet UV light. The vision in the right eye got cloudy and the color perception between the right eye and the left eye differed. In addition the perceived size of objects seemed to differ between the two eyes.

The left eye was exposed to a red colored LED, with the eyelid closed. Visible red light could be seen through and illuminating the eyelid. With the exception of certain positions of the LED relative to the right eye, the right eye had difficulty in perception of the red light. The right eye was exposed to periodic exposures of light from a red colored LED. Improvements to the right eye were noted on a daily basis. Within six months, the right eye appeared to be 90% healed. After six months of daily exposures to the red colored LEDm the perception of the left eye and the right eye are almost identical.

It should be understood that the example and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein (including those in the “References” section) are incorporated by reference in their entirety, including all figures and tables, to the extent they are not inconsistent with the explicit teachings of this specification.

What is claimed is:

1. A low-level laser light therapy (LLLLT) device, comprising:
a pulse generator configured to generate a pulse train,
wherein a pulse wave of the pulse train is configured to have a rectangular, non-sinusoidal waveform and a duty cycle within a range of 0.5% and 20%;
an emitter array comprising a plurality of light emitting diodes (LEDs) and in electrical connection with the pulse generator,
a computer-readable medium comprising stored instructions which, when executed, causes an LED to emit light; and
a housing unit.

2. The device of claim 1, wherein the pulse wave of the pulse train has a duty cycle of 1.5% to 3.5%.

3. The device of claim 1, wherein at least one LED is configured to output light at a pulse repetition frequency within a range of 55 kHz to 70 kHz.

4. The device of claim 1, wherein at least one LED is configured to output light at a subharmonic of a pulse repetition frequency of 65 kHz.

5. The device of claim 1, wherein at least one LED is configured to output light at a pulse repetition frequency of 125 kHz.

6. The device of claim 1, wherein a pulse wave has a period of 15.13 μs.

7. The device of claim 1, wherein a pulse wave has a period that is an integer multiple or submultiple of 15 μs.

8. The device of claim 1, wherein the emitter array comprises a first LED and a second LED, wherein the LED is configured to emit light at a first wavelength within a first specified range and the second LED is configured to emit light at a second wavelength within a second specified range, wherein the first specified range is different than the second specified range.

9. The device of claim 1, wherein the computer-readable medium comprising stored instructions which, when executed, further causes an LED to emit a predetermined number of light emissions.

10. The device of claim 1, wherein the computer-readable medium comprising stored instructions which, when executed, further causes an LED to emit light for a predetermined period of time.

11. The device of claim 1, wherein the LEDs are arranged in an N×M pattern, wherein N or M can be any value of 1 or greater.

12. The device of claim 1, wherein four LEDs are arranged in a square pattern, wherein each LED is disposed at a corner of the square pattern, and wherein a fifth LED is disposed at a center of the square pattern.

13. The device of claim 1, wherein the housing unit has a first face, positioned on a first side of the device that comprises: an “on” switch configured to turn the device on; an “off” switch configured to turn the device off; a “momentary” switch configured to turn the device on when depressed and to turn the device off when released; a LED configured to emit visible light when the device is on and not emit visible light when the device is off; and a second face positioned on a second side, wherein the second face comprises a plurality of apertures configured to permit the emitter array to emit light.

14. The device of claim 13, wherein the second face is parallel to and on an opposite side of the first face.

15. The device of claim 13, wherein the second face is perpendicular to the first face.

16. A low-level laser light therapy (LLLLT) device, comprising:
a first module comprising:
a control interface having a plurality of operable switches configured to produce an LED regulating signal,
wireless transmitter circuitry configured to encode the LED regulating signal and wirelessly transmit the encoded LED regulating signal; and
at least one second module comprising:
a pulse generator or repeater configured to generate a pulse train,
wherein a pulse wave of the pulse train is configured to have a duty cycle between 0.5% and 20%,
at least one emitter array,
wherein the at least one emitter array comprises a plurality of LEDs, and
wherein at least one LED is configured to operate in response to the decoded LED regulating signal;
wireless receiver circuitry configured to receive the encoded LED regulating signal, decode the encoded LED regulating signal, and transmit the decoded LED regulating signal to the emitter array; and
a housing unit to house the at least one emitter array.

17. The device of claim 16, wherein at least one LED is configured to emit light at a wavelength within a range of 280 nm to 570 nm to stimulate cellular regeneration in plants.

18. A method of providing low-level laser light therapy (LLLT) to a patient need, the method comprising:
providing a low-level laser light therapy device as described in claim 16;
positioning the device at a distance from a target region of a patient in need; and
exposing the target region of the patient in need to the emit light;
wherein the pulse repetition frequency and the wavelength of the emitted light is configured for the treatment of at least one of the following:
hypertrophic scarring;
keloid scar tissue;
breathing and respiratory problems;
bone to bone knee issues;
joint pain;
calcification of bone;
abdominal pain;
Irritable Bowel Syndrome;
edema;
hair loss;
Seasonal Affective Disorder;
sleep disorder;
sciatica;
cataracts; and
eye injury.

19. The device of claim 16, wherein the device is configured to modulate any emitted light in order to simulate the seasons or in response to seasonal changes.

20. A method of promoting plant growth, wherein said method comprises:
outputting light at a pulse repetition frequency of 125 kHz from an LED of the device of claim 1;
positioning the device at a distance from a target region of a subject plant in need; and
exposing the target region of the subject plant in need to the output light.
